(19) United States
(12) Patent Application Publication
(10) Pub. No.: US 2004/0131145 A1
(43) Pub. Date: Jul. 8, 2004

(54) Mammography apparatus

(75) Inventor: Hiromu Ohara, Tokyo (JP)

Correspondence Address: FRISHAUF, HOLTZ, GOODMAN & CHICK, PC, 767 THIRD AVENUE, 25TH FLOOR, NEW YORK, NY, 10017-2023, US

(73) Assignee: KONICA MINOLTA HOLDINGS, INC., Tokyo (JP)

(21) Appl. No.: 10/741,550

(22) Filed: Dec. 18, 2003

(30) Foreign Application Priority Data
Dec. 27, 2002 (JP) 2002-378937

(51) Int. Cl. A61B 6/04

(52) U.S. Cl. 378/37

(57) ABSTRACT

A mammography apparatus capable of irradiating radiation having enough irradiation dose to radiograph an image surely. The mammography apparatus has: a radiation source; a subject platform for supporting a subject so as to face the subject to the radiation source; a radiation image detector located so as to be faced to the radiation source with respect to the subject platform for detecting radiation transmitted through the subject; a controller for controlling the radiation source; wherein the controller sets an irradiation condition of the radiation to be irradiated from the radiation source based on control conditions including at least thickness of the subject and a distance from the radiation source to the subject platform.

### BACKGROUND OF THE INVENTION

1. Field of the Invention

This invention relates to a mammography apparatus, in particular a mammography apparatus capable of radiographing a phase contrast image.

2. Description of Related Art

Conventionally, as an apparatus for an image diagnosis in a medical field, a mammography apparatus is available. The mammography apparatus is to obtain radiation image information by irradiating radiation which is at a level of not harming health of an examinee, from a radiation source toward a subject, which is a mamma of the examinee, and detecting the radiation transmitted through the subject with a radiation image detector (for example, see Japanese Patent Application Publication (Unexamined) No. Tokukai 2001-238871).

The mammography apparatus comprises a controller for controlling irradiation dose of radiation irradiated by the radiation source. Concretely, the controller controls irradiation dose from the radiation source based on the irradiation dose of radiation detected by a radiation dose detector, which is the so-called phototimer. The phototimer is fixedly placed at a side opposite to the radiation source with respect to the radiation image detector, and capable of detecting amount of radiation transmitted through the subject and the radiation image detector, that is, transmitted radiation dose.

However, in the mammography controlling the irradiation dose based on the above-mentioned phototimer, for example, if a location of the subject is deviated from a location just above the phototimer, or if the subject is not capable of being located just above the phototimer because the subject is too small, transmitted radiation dose through the subject is not detected accurately. Therefore, in the above-mentioned cases, sometimes it is not possible to detect radiation with the radiation image detector because radiation having suitable irradiation dose is not irradiated.

### SUMMARY OF THE INVENTION

An object of the present invention is to provide a mammography apparatus capable of irradiating radiation having enough irradiation dose to radiograph an image surely.

In accordance with a first aspect of the present invention, a radiation source; a subject platform for supporting a subject so as to face the subject to the radiation source; a radiation image detector placed so as to be faced to the radiation source with respect to the subject platform for detecting radiation transmitted through the subject; a controller for controlling the radiation source; wherein the controller sets an irradiation condition of the radiation to be irradiated from the radiation source based on control conditions including at least thickness of the subject and a distance from the radiation source to the subject platform.

Here, the subject is a mamma of the examinee.

Further, the irradiation condition of radiation is to determine irradiation dose of radiation to be irradiated from the radiation source, that is, exposure dose of the subject. As such an irradiation condition, other than the above-mentioned irradiation dose itself, for example, a tube voltage to be impressed to a target (rotating anode) in a radiation tube (radiation source) or the like. Here, the target in the radiation tube is a part for generating radiation in response to the voltage impression.

Further, the distance from the radiation source to the subject platform and the thickness of the subject are respectively ones of parameters for changing exposure dose of the subject. According to these values, it is possible to estimate exposure dose of the subject approximately accurately.

According to the mammography apparatus, since the irradiation condition of radiation to be irradiated from the radiation source is set based on the control conditions comprising the distance from the radiation source to the subject platform, the thickness of the subject and the like, it is possible to estimate exposure dose of the subject from the control conditions without using a phototimer, and to set the irradiation condition of radiation based on the estimation result. Therefore, since the irradiation condition is set without using a phototimer unlike the conventional art, it is possible to surely prevent from irradiating radiation having so small irradiation dose that the radiation image detector cannot detect the radiation, regardless of the position of the subject on the subject platform. In other words, it is possible to irradiate radiation having enough irradiation dose to radiograph an image surely. Further, it is possible to prevent from irradiating excessive amount of radiation to the examinee.

Preferably, in the apparatus of the first aspect of the present invention, the control conditions include an age of an examinee.

Here, the age of the examinee is one of the parameters for changing the exposure dose of the subject and it makes the exposure dose of the subject estimable more accurately by being used with the distance from the radiation source to the subject platform and the thickness of the subject.

Thereby, since the irradiation condition of radiation to be irradiated from the radiation source is set based on the distance from the radiation source, the thickness of the subject and the age of the examinee, it is possible to estimate the exposure dose of the subject more accurately. Therefore, it is possible to irradiate radiation having enough irradiation dose to radiograph an image more surely.

Further, in the mammography apparatus of the present invention, preferably, an age input device for inputting the age of the examinee is connected to the controller, and the age input device outputs the input age to the controller.

Thereby, since it is possible to input the age of the examinee through the age input device, it is possible to obtain the same effect as the above-mentioned invention.

Here, the age input device may be either integrated with, or placed separately from the controller.

Further, in the mammography apparatus of the present invention, preferably, the control conditions include density information regarding density of the subject.

Here, the density information is, for example, a ratio of mammary gland and fat in the subject or the like. The density information is one of the parameters for changing the exposure dose of the subject, and it makes the exposure dose of the subject estimable more accurately by being used with the distance from the radiation source to the subject platform and the thickness of the subject.

Thereby, since the irradiation condition of radiation to be irradiated from the radiation source is set based on the distance from the radiation source to the subject platform, the thickness of the subject and the density information, it is possible to estimate the exposure dose of the subject more accurately. Therefore, it is possible to irradiate radiation having enough irradiation dose to radiograph an image more surely.

Further, in the mammography apparatus of the present invention, preferably, a density information input device for inputting the density information is connected to the controller, and the density information input device outputs the input density information to the controller.

Thereby, since it is possible to input the density information through the density information input device, it is possible to obtain the same effect as the above-mentioned invention.

Here, the density information input device may be either integrated with, or placed separately from the controller.

Preferably, the mammography apparatus of the present invention further comprises a pressure plate for compressing the subject supported by the subject platform in conjunction with the subject platform, a measuring device for measuring a distance from an under surface of the pressure plate to an upper surface of the subject platform, wherein the controller uses a result of the measurement by the measuring device as the thickness of the subject.

Thereby, it is possible to measure the thickness of the subject as the distance from the under surface of the pressure plate to the upper surface of the subject platform. Therefore, it is possible to prevent from the thickness of the subject of the identical examinee changing depending on the operator, and to measure the thickness of the subject accurately. Therefore, it is possible to estimate the exposure dose of the subject more accurately, and thereby it is possible to irradiate radiation having enough irradiation dose to radiograph an image even more surely.

Further, in the mammography apparatus of the present invention, preferably, the thickness of the subject is the result of the measurement by the measuring device when the pressure plate compresses the subject in conjunction with the subject platform with a predetermined pressure.

Thereby, since the result of the measurement by the measuring device is used as the thickness of the subject when the subject is compressed with a predetermined pressure, it is possible to prevent from the thickness of the subject of the identical examinee changing depending on the operator surely, and to measure the thickness of the subject more accurately. Therefore, it is possible to estimate the exposure dose of the subject more accurately, and to irradiate radiation having enough irradiation dose to radiograph an image even more surely.

Further, in the mammography apparatus of the present invention, preferably, the controller comprises a first storage section for correspondingly storing the irradiation condition to be set and each of the control conditions as a data table in advance, and sets the irradiation condition by referring to the data table based on the control conditions.

Thereby, since the controller comprises the first storage section for storing the irradiation condition to be set and each of the control conditions as the data table in advance where the irradiation condition and each of the control condition are related to each other, and sets the irradiation condition with reference to the data table based on the control conditions, it is possible to estimate the exposure dose of the subject immediately, and to irradiate radiation having enough irradiation dose to radiograph an image surely.

Further, in the mammography apparatus of the present invention, preferably, the irradiation condition includes at least one of irradiation dose of the radiation and voltage impressed to the radiation source.

Thereby, by setting at least one of the irradiation dose of radiation and the voltage impressed to the radiation source as the irradiation condition, it is possible to obtain the same effect as the above-mentioned invention.

Further, in the mammography apparatus of the present invention, preferably, the controller comprises a second storage section for correspondingly storing subject identification information for identifying the subject and the set irradiation condition, and the controller uses the irradiation condition stored in the second storage section in advance as the irradiation condition of the radiation to be irradiated when the identical subject is to be radiographed at the second time or more.

Thereby, when the identical subject is to be radiographed at the second time or more, since the irradiation condition stored in the second storage section in advance as the irradiation condition of radiation to be irradiated is used, radiation having the same irradiation dose is always irradiated to the identical subject at any time of radiography. Therefore, since it is possible to stabilize the irradiation dose to the identical subject, it is possible to surely prevent from irradiating radiation having so small irradiation dose that the radiation image detector cannot detect the radiation. In other words, it is possible to irradiate radiation having enough irradiation dose to radiograph an image surely. Further, it is possible to prevent from irradiating excessive amount of radiation to the examinee.

Further, preferably, the mammography apparatus of the present invention preferably further comprises a notifying section for notifying an operator of abnormality of irradiation dose of the radiation to be irradiated, wherein the controller comprises a second storage section for correspondingly storing subject identification information for identifying the subject and the set irradiation condition in advance, and is connected to the notifying section, and when the identical subject is to be radiographed at the second time or more and irradiation dose of the radiation to be irradiated based on the set irradiation condition is more than predetermined amount with respect to irradiation dose of the radiation based on the irradiation condition stored in the second storage section in advance, the controller has the notifying section notify the operator accordingly.

Thereby, when irradiation dose of radiation to be irradiated is more than a predetermined amount with respect to the irradiation dose of radiation irradiated in the past, the operator is notified accordingly. Consequently, it is possible to prevent from irradiating excessive amount of radiation to the examinee.

Further, in the mammography apparatus of the present invention, preferably, a distance from the radiation source to the radiation image detector is settable from 75 cm to 200 cm, and a distance from the subject platform to the radiation image detector is settable from 15 cm to 100 cm.

Here, in order to radiograph a clear phase contrast image, it is necessary that a distance from the radiation source to the radiation image detector be not less than 75 cm, and a distance from the subject platform to the radiation image detector be not more than 15 cm. The larger these distances become, the more preferable.

However, if the distance from the radiation source to the radiation image detector becomes too large, the whole size of the mammography apparatus increases, and thereby its usability decreases. Therefore, preferably the above-mentioned distance is not more than 200 cm in view of usability.

Further, if the distance from the subject platform to the radiation image detector becomes too large compared to the distance from the radiation source to the subject platform, sharpness of an image decreases due to influence of the so-called half shadow, that is, unclearness. Therefore, preferably, the distance from the subject platform to the radiation image detector is not too large compared to the distance from the radiation source to the subject platform. Here, the half shadow is a phenomenon in which one point on the subject is detected as a figure having a size on the radiation image detector due to the focus size.

Thereby, since the distance from the radiation source to the radiation image detector is not less than 75 cm and the distance from the subject platform to the radiation image detector is not less than 15 cm, it is possible to radiograph a clear phase contrast image.

Further, since the distance from the radiation source to the radiation image detector is not more than 200 cm, it is possible to improve the usability of the mammography apparatus.

Further, since the distance from the subject platform to the radiation image detector is not more than 100 cm, the distance from the subject platform to the radiation image detector is not too large compared to the distance from the radiation source to the subject platform. Therefore, it is possible to radiograph a sharp image with little influence of unclearness.

Further, in the mammography apparatus of the present invention, preferably, the distance from the radiation source to the radiation image detector is settable from 100 cm to 160, and the distance from the subject platform to the radiation image detector is settable from 25 cm to 80 cm.

Thereby, since the distance from the radiation source to the radiation image detector is not less than 100 cm and the distance from the subject platform to the radiation image detector is not less than 25 cm, it is possible to radiograph a sharper phase contrast image.

Further, since the distance from the radiation source to the radiation image detector is not more than 160 cm, it is possible to improve the usability of the mammography apparatus more.

Further, since the distance from the subject platform to the radiation image detector is not more than 80 cm, the distance from the subject platform to the radiation image detector is not too large compared to the distance from the radiation source to the subject platform. Therefore, it is possible to radiograph a sharp image with little influence of unclearness.

Further, in the mammography apparatus of the present invention, preferably, the radiation image detector is a photostimulable phosphor plate or a flat panel detector.

Thereby, since the radiation image detector is a photostimulable phosphor plate, with the property of accumulative phosphor (photostimulable phosphor) emitting stimulated light according to energy of radiation irradiated to the subject, it is possible to record radiation image information of the subject and to provide an image of the subject.

Further, since the radiation image detector is a flat panel detector, it is possible to record radiation image information of the subject by detecting intensity of radiation irradiated to the subject, and to provide an image of the subject.

Further, in the mammography apparatus of the present invention, preferably, a focus size of the radiation source is from 50 μm to 250 μm.

Here, if the focus of the radiation source is a square, length of its side is defined as the focus size. If the focus is a circle, its diameter is defined as the focus size, and if the focus is a rectangle, its short side is defined as the focus size.

Thereby, since the focus size is not less than 50 μm, it is possible to have large current on the radiation source. Therefore, since it is possible to obtain irradiation dose (=current×irradiation period) necessary to perform radiography despite a short time period, it is possible to prevent from an image getting unclear due to the movement of organs in the examinee's body. In other words, it is possible to radiograph a sharp image with little unclearness.

Further, since the focus size is not more than 250 μm, in the case of magnified radiography, unclearness of the half shadow hardly happens. Therefore, it is possible to radiograph a sharp image with little unclearness.

### BRIEF DESCRIPTION OF THE DRAWINGS

The present invention will become more fully understood from the detailed description given hereinafter and the accompanying drawing given by way of illustration only, and thus are not intended as a definition of the limits of the present invention, and wherein:

FIG. 1 is a schematic view showing main components of a mammography apparatus to which the present invention is applied, and

FIG. 2 is a block diagram showing main components of a controller.

### AN EMBODIMENT OF THE INVENTION

Hereinafter, an embodiment of the present invention will be explained with reference to figures.

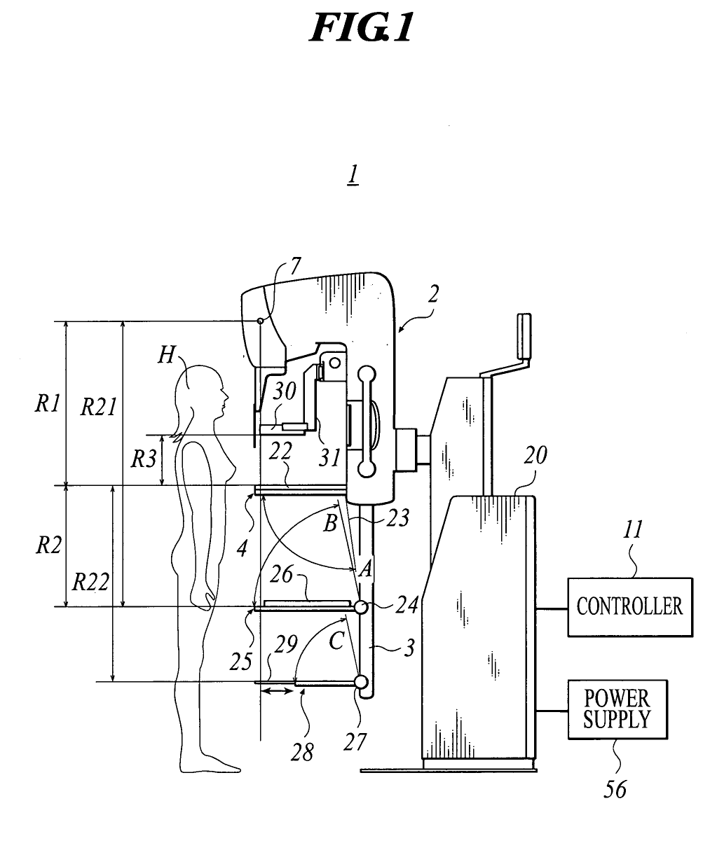

FIG. 1 is a schematic view showing main components of a mammography apparatus 1 of the present embodiment to which the present invention is applied. Here, the mammography apparatus 1 radiographs an absorption contrast image in a normal radiography mode, and radiographs a phase contrast image in a phase contrast image radiography mode.

In the mammography apparatus 1, a radiography unit 2, having a shape of a letter “7” when it is seen from its side, is placed while being supported by a support base 20 having a pillar shape.

At a lower part of the radiation unit 2, placed are a subject platform 4 projecting horizontally for supporting a subject H, and a support axis 3 projecting downwardly for supporting a radiation image detector 26 and the like. At an upper part of the radiography unit 2, placed is a radiation source 7 for irradiating radiation toward the subject platform 4.

The subject platform 4 is capable of moving vertically, and an interval R1 between the subject platform 4 and the radiation source 7 satisfies an equation: 50 [cm]≦R1≦=100 [cm]. The interval R1 is one of parameters for changing exposure dose of the subject H, and one of control conditions for controlling irradiation dose of the radiation source 7.

Above the subject platform 4, a pressure plate 30 having a plate shape is placed approximately in parallel with the subject platform 4. The pressure plate 30 is capable of moving vertically along a rail (a measuring device) 31 which is placed in the radiography unit 2 as extending vertically, and compresses the subject H supported on the subject platform 4 from above with pressure from 50 to 150 [N], for example, 100 [N].

The rail 31 also functions as an ohmmeter, and detects an ohmic value according to a location of the pressure plate 30. Accordingly, an interval R3 between an under surface of the pressure plate 30 and an upper surface of the subject platform 4, that is a thickness of the subject H when the subject H is sandwiched between the pressure plate 30 and the subject platform 4. The interval R3 is one of the parameters for changing exposure dose of the subject H, and one of the control conditions for controlling the irradiation dose of the radiation source 7.

Further, beneath the subject platform 4, placed is a radiation image detector 23 to be used in the normal radiography mode. The radiation image detector 23 is capable of rotating from a horizontal position to a position A with a first rotation axis as its center. In the normal radiography mode, the radiation image detector 23 is set at the horizontal position, and in the phase contrast image radiography mode, it is set at the position A. In detail, the radiation image detector 23 is set on the upper surface of the subject platform 4 while being at the horizontal position, in other words, it is set so as to have no interval between the subject H and the radiation image detector 23. Further, while being at the position A, the radiation image detector 23 is away from an irradiation field of the radiation source 7. Here, the radiation image detector 23 may be placed as detachable from the subject platform 4.

The support axis 3 is placed as extending vertically. At the support axis 3, placed are supporting platforms 25 and 28 for respectively supporting radiation image detectors 26 and 29 to be used in the phase contrast image radiography mode detachably.

The supporting platform 25 is located below the subject platform 4, and capable of rotating from a horizontal position to a position B with a second rotation axis 24 as its center.

When the supporting platform 25 is at the horizontal position, it is possible to radiograph a phase contrast image. At this time, an interval R2 between the radiation image detector 26 supported by the supporting platform 25 and the upper surface of the subject platform 4 satisfies an equation: 15 [cm]≦R2≦100 [cm], preferably 25 [cm]≦R2≦80 [cm]. Further, an interval R21 between the radiation source 7 and the radiation image detector 26 supported by the supporting platform 25 satisfies an equation: 75 [cm]≦R21≦200 [cm], preferably 100 [cm]≦R21≦160 [cm].

In the normal radiography mode or when the radiation image detector 29 is used, the radiation image detector 26 is detached from the supporting platform 25. At this time, the supporting platform 25 moves up to the position B so as to be away from the irradiation field of the radiation source 7.

The supporting platform 28 is located below the supporting platform 25, and capable of rotating from a horizontal position to a position C with a third rotation axis 27 as its center. Further, the supporting platform 28 is slid at the horizontal position so as to extend, and is slid at the position C so as to contract.

The radiation image detector 29 is detached from the supporting platform 28 in the normal radiography mode or when the radiation image detector 26 is used. At this time, the supporting platform 28 is slid so as to contract and moved up to the position C.

Here, an interval R22 between the upper surface of the subject platform 4 and the radiation image detector 29 is set so as to be larger than the interval R2 between the upper surface of the subject platform 4 and the radiation image detector 26. Therefore, it is possible to obtain an image with a higher magnifying rate when radiography is performed with the radiation image detector 29 than when radiography is performed with the radiation image detector 26.

Here, relation between the interval R21, which is between the radiation source 7 and the radiation image detector 26, and the interval R2, which is between the subject platform 4 and the radiation image detector 26, will be explained. While it is preferable to have the R2 larger than the R21 because larger edge effect due to phase contrast is expected, if R2 is too large compared to R21, sharpness decreases due to influence of unclearness of half shadow. Accordingly, in view of improving image quality, both R21 and R2 are large, desirably. However, in an actual case where a mammography apparatus is used in a radiography room, in consideration of cases of rotating the apparatus when it is used, and relation of the size of the radiography room and the size of the apparatus, it is inconvenient to use the apparatus being too large. Therefore, in view of image quality and convenience, desirable sizes of the above-mentioned R21 and R2 are determined.

Next, the radiation image detectors 23, 26 and 29 will be explained. The radiation image detectors 23, 26 and 29 are to detect radiation transmitted through the subject H. Concretely,

A. a combination of radiation fluorescent intensifying screen and silver halide photographic film,

B. a photostimulable phosphor plate emitting light with photo-stimulation,

C. a radiation image information reading apparatus having scintillators for converting radiation energy into light and light semiconductor devices for reading the light, arrayed two-dimensionally,

D. a radiation image information reading apparatus having photoconductors for directly converting radiation energy into electric signals and semiconductor devices for reading the electric signals, arrayed two-dimensionally,

E. a radiation image information reading apparatus having either a single or a plurality of combinations of scintillators for converting radiation into light and lenses for light-focusing the light to CCD, CMOS or the like arrayed, and

F. a radiation image information reading apparatus having scintillators for converting radiation into light and replacing the light with electric signals by leading the light to CCD, CMOS or the like with optical fiber,

can be used.

Here, a combination of radiation fluorescent intensifying screen and silver halide photographic film as mentioned in A applied to the radiation image detectors 23, 26 and 29 is also called an SF system (Screen Film system). Radiation fluorescent intensifying screen has rare-earth phosphor such as calcium tungstate, gadolinium oxy-sulphide or the like, and replaces radiation energy with either blue or green luminescence. In particular, regarding intensifying screen using rare-earth phosphor, a technique disclosed in Japanese Patent Application Publication (Unexamined) No. Tokukai-hei 6-67365 may be used. Further, as the silver halide photographic film, preferably the one having either a single or both sides of a supporting body coated with photosensitive emulsion is used. Especially, in the case of using duplicated film, preferably photographic material in which photographic characteristic is different among each of the emulsion layers over the film supporting body is used. Further, preferably, photographic film having a layer for absorbing crossover light placed between each emulsion surface of the duplicated film is used. In the present embodiment, a size of either single-sided and/or duplicated film can be any, from dividing-into-six size to half-dividing size. The silver halide photographic sensitive material is explained in Japanese Patent Application Publication (Unexamined) No. Tokukai-hei 6-67365 or, for example, “Revised Basis of Photography Engineering—Edition of Silver Halide Photography—” (edited by The Society of Photographic Science and Technology of Japan, published by Corona Publishing Co., Ltd.). Further, regarding film processing of the photographic film, although it is possible to improve average tone by raising film processing temperature or extending a time period for the film processing, preferably a film processing condition assigned by a film manufacturer essentially is used when automatic film processing is done.

With the photostimulable phosphor as mentioned in B, by irradiating visible light after the irradiation, visible light luminescence corresponding to intensity of the radiation which already has been irradiated is inducted. In other words, after the radiation is irradiated, the photostimulable phosphor is moved to a laser reading apparatus for reading emitted light, and the read light is replaced with electric signals by use of an electron multiplier for obtaining the electric signals of the radiation image. The electric signals are, after being applied appropriate image processes on, either displayed on an image display unit such as a monitor or the like, and/or output as hardcopy of the radiation image by use of an image output unit such as a laser imager or the like. At this time, if the image has been magnified, it is possible to either display it on the monitor and/or output as hardcopy by reducing it back to substantially life size with a predetermined magnifying rate input in advance. Regarding the radiation image detector using the photostimulable phosphor, a technique for visualizing images such as a phosphor, reading emitted light or the like disclosed in Japanese Patent Application Publication (Laid-open) No. Tokugan 2000-245721, is preferably used in the present embodiment.

Regarding the reading apparatuses for converting radiation into electrical signals as mentioned in C to F, techniques disclosed in “Handbook of Medical Imaging” Vol. 1, chapter 4 “Flat Panel Imagers for Digital Radiography” (ed. R. V. Matter et al. SPIE Press, Bellingham, 2000) are preferably used in the present embodiment. In these cases, the electrical signals of the radiation image obtained at the radiation image detector 23, 26 and 29 are appropriately processed and their images are drawn on a monitor or a hardcopy to be used for an image diagnosis or the like.

In the case of using the radiation image detector to which these C to F are applied, a conventional radiation image detector having a size of 18 [cm]×24 [cm], or 24 [cm]×30[cm] is used. On the other hand, in the phase contrast image radiography mode, since it is magnifying radiography, preferably one having a large size is used. Concretely, the size of the radiation image detector is larger than 25 [cm]×32 [cm], and in consideration of easy handling, preferably the size is approximately 35 [cm]×43 [cm].

As the radiation source 7, a radiation tube having a focus size of 300 [μm] and a radiation tube having a focus size of 100 [μm] are placed as capable of switching each other. Concretely, the radiation tube with focus size 300 [μm] is used in the normal radiography mode, and the radiation tube with focus size 100 [μm] is used in the phase contrast image radiography mode. Here, the focus of the radiation source is, for example, a window for taking radiation generated by crashing electrons into a rotating anode of the radiation tube. Generally, if the focus is a square, and length of its side is defined as a focus point size D. If the focus is a circle, its diameter is defined as the focus point size D. If the focus is a rectangle, its short side is defined as a focus point size D. As a measuring method of the focal point size D, a method with a pinhole camera, a method with a microtest chart and the like are written in JIS Z 4704.

Further, as these radiation tubes, a radiation tube irradiating radiation having wavelength of 0.01 to 0.1 [nm] is used. In such a radiation tube, as a result of accelerating electrons generated from thermal excitation with high voltage and crashing them into an anode, kinetic energy of the electrons are converted into radiation energy for irradiating radiation from the focus. At the time of radiographing a radiation image, the accelerating voltage is set as tube voltage, amount of generated electrons is set as tube current and an radiation irradiating period is set as an exposure period. The anode (anticathode) is made of copper, molybdenum, rhodium, tungsten or the like. Depending on a kind of the anode, energy spectrum of irradiated radiation changes. In the present embodiment, the anode of copper, molybdenum, rhodium or the like among these anodes is used, and therefore it is possible to obtain line spectrum having narrow radiation energy distribution and comparatively low energy. Further, the anode is the so-called rotating anode, which rotates so as to spread locations into which electrons crash. Since it is possible to spread locations where heat is developed due to the crash of electrons, such an anode have an advantage of being difficult to melt.

The support base 20 comprises a power supply 56 and a controller 11 for controlling operation of the radiography unit 2.

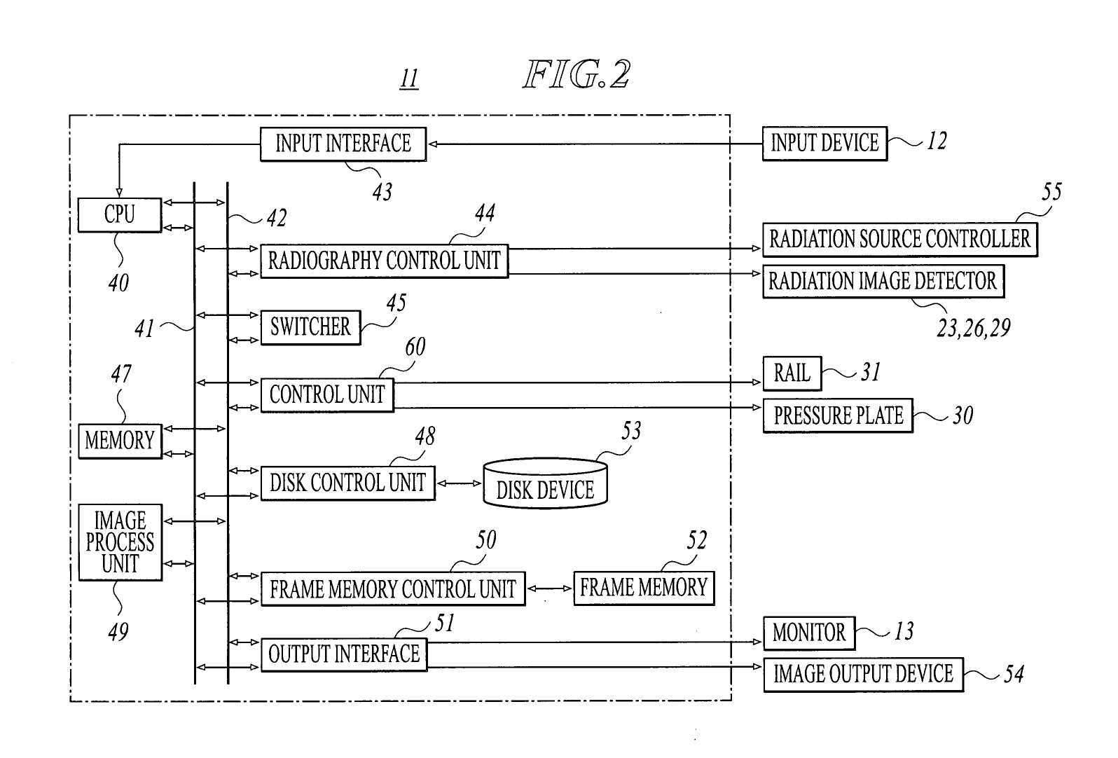

As shown in FIG. 2, the controller 11 comprises a CPU 40 for controlling all the operation of the controller 11. Connected to the CPU 40 are a system bus 41, an image bus 42 and an input interface 43, and the CPU 40 controls operation of each unit via the system bus 41 and radiation image information is transmitted between each unit via the image bus 42. Connected to the system bus 41 and the image bus 42 are a radiography control unit 44, a switcher 45, a frame memory control unit 50, a disk control unit 48, an output interface 51, an image process unit 49, the input interface 43, a memory 47, a control unit 60 and the like.

The radiography control unit 44 reads radiation image information from the radiation image detectors 23, 26 and 29 and supplies it to the frame memory control unit 50. Further, the radiography control unit 44 rotates the radiation image detector 23 from its horizontal position to the position A in the phase contrast image radiography mode, rotates the supporting platform 25 from its horizontal position to the position B when the supporting platform 25 does not support the radiation image detector 26, and rotates the supporting platform 28 from its horizontal position to the position C when the supporting platform 28 does not support the radiation image detector 29.

The switcher 45 is a means to switch between the phase contrast image radiography mode and the normal radiography mode. Here, instruction of the switch may be input at an input device 12 of the input interface 43.

Connected to the frame memory control unit 50 is a frame memory 52, and the frame memory 52 stores radiation image information generated in the radiation image detectors 23, 26 and 29. The radiation image information stored in the frame memory 52 is read out and supplied to the disk control unit 48. The frame memory 52 may store radiation image information which has been processed by the image process unit 49 after being supplied from the radiation image detectors 23, 26 and 29.

When the frame memory 52 supplies radiation image information to the disk control unit 48, the radiation image information is continuously read out and written in a FIFO memory in the disk control unit 48. After that, the radiation image information is sequentially stored in a disk device 53. The disk device 53 is capable of storing the radiation image information stored in the frame memory 52, that is, radiation image information which has been processed by the image process unit 49 after being supplied from the radiation image detectors 23, 26 and 29, along with administrative information and the like. Here, the administrative information includes, for example, subject identification information for identifying the subject H, information for identifying radiation image information, information regarding radiography such as a magnifying rate of a radiation image, or the like.

The radiation image information read out from the frame memory 52 and the radiation image information read out from the disk device 53 are supplied to an image output device or a monitor (a notifying unit) 13 as an image output means, to be provided to a user as a visible image. The monitor 13 notifies an operator of abnormality of irradiation dose of radiation to be irradiated.

The image process unit 49 performs an irradiation field recognition process, a setting of region of interest, a normalization process, a gradation process and the like of the radiation image information supplied from the radiation image detectors 23, 26 and 29 via the radiography control unit 44. Further, the image process unit 49 performs a frequency emphasis process, a dynamic range compression process and the like. Further, the image process unit 49 is capable of automatically reducing an image radiographed with a magnifying rate larger than one, back into a life-size image based on the radiography mode information, and either displaying and/or outputting the image at approximately life size through the monitor 13 and/or the image output device 54. Here, the CPU 40 may have the function of the image process unit 49 for performing the image process and the like.

Input to the input interface 43 are the interval R3 between the under surface of the pressure plate 30 and the upper surface of the subject platform 4 from the rail 31, radiation intensity information and image electronic signals from the radiation image detectors 23, 26 and 29, and the radiography mode information from the switcher 45. Further, information such as sensitivity of the radiation image detector, a set voltage value of the radiation tube and the like is input to the input interface 43. Connected to the input interface 43 is the input device (an age input device, a density information input device) 12 such as a keyboard or the like.

The operator inputs the administrative information to the input device 12. Here, the input of the administrative information is made by use of not only the keyboard, but also a magnetic card, a barcode, an HIS (information administration according to Hospital Information System network) or the like.

Further, the input device 12 comprises a switch for inputting an age of the examinee and a switch for selecting and inputting density information regarding density of the subject H. By use of these switches, the operator inputs the age of the examinee and the density information. Here, the age and the density information respectively are ones of the parameters for changing exposure dose of the subject H, and ones of the control conditions for controlling irradiation dose of the radiation source 7. Further, the density information is, for example, an evaluation figure concerning a ratio of mammary gland and fat, and is determined by the operator based on examinee's physique or the like. In the present embodiment, the density information is expressed at three degrees “high”, “middle” and “low”.

When each of the control conditions of the interval R1 between the radiation source 7 and the subject platform 4, the interval R3 between the under surface of the pressure plate 30 and the upper surface of the subject platform 4, the density information of the subject H, and the age of the examinee is a predetermined standard control condition, the memory (a first storing unit, a second storing unit) 47 stores the standard control condition of the radiation source 7. Concretely, the standard control condition of the interval R1 between the radiation source 7 and the subject platform 4 is 65 [cm], the standard control condition of the interval R3 between the under surface of the pressure plate 30 and the upper surface of the subject platform 4 is 4 [cm], the standard control condition of the density of the subject H is “middle” and the standard control condition of the age of the examinee is 50 [years old]. Further, as standard irradiation conditions under the above-mentioned standard control conditions, standard tube voltage to be impressed to the radiation tube is 28 [kVp] and standard irradiation dose of the irradiation dose is 50 [mAs] (here, irradiation dose [mAs]=current amount [mA]×time [Sec]). Here, the value of the standard irradiation dose is an example, and therefore the value will change depending on a mammography apparatus and a difference of a facility.

Further, in the memory 47, a first data table, a second data table and a third data table showing relation between each of the above-mentioned control conditions and a ratio value of irradiation dose of radiation to be irradiated from the radiation source 7 with respect to the above-mentioned standard irradiation dose. These first data table, second data table and third data table are respectively shown in Graph 1, Graph 2 and Graph 3.

Graph 1:

| mamma | | | | | | | 7 cm and |
|---|---|---|---|---|---|---|---|
| thickness | 1 cm | 2 cm | 3 cm | 4 cm | 5 cm | 6 cm | higher |
| ratio value | 0.72 | 0.81 | 0.90 | 1.00 | 1.11 | 1.23 | 1.37 |

Graph 2:

| | density | | |
|---|---|---|---|
| | high | middle | low |
| ratio value | 1.15 | 1.00 | 0.87 |

Graph 3:

age

| | 30 and | | | 70 and |
|---|---|---|---|---|
| | lower | 30 s and 40 s | 50 s and 60 s | higher |
| ratio value | 1.1 | 1.05 | 1 | 0.95 |

As shown in Graphs 1 to 3, when the interval R3 between the under surface of the pressure plate 30 and the subject platform 4 is large, irradiation dose of radiation to be irradiated increases. Further, when the age of the examinee is high, irradiation dose of radiation to be irradiated decreases.

The above-mentioned first to third data tables can be generated by measuring accrual amount of impressed tube voltage, a size of an aluminum half value layer at each tube voltage value, amount of exposure dose of the subject H at each distance from the radiation source 7 at certain irradiation amount, thickness of the subject H, density of the subject H and the like, in advance. Here, the size of an aluminum half value layer is, thickness [mm] of an aluminum layer when transmitting radiation dose is cut half. Regarding a method for measuring each value as above mentioned to be used for setting irradiation dose, it is possible to refer to “a manual for breast cancer examination with mammography—precision management manual” (Japan Medical Journal, 2002), “mammography precision management manual (revised)” (Japanese Society of Radiological Technology, 1999) and the like.

Further, the memory 47 stores the subject identification information and the irradiation conditions configured in the past correspondingly. Here, these subject identification information and irradiation conditions may be stored in the disk device 53, or a storage device other than the controller 11.

The control unit 60 lowers the pressure plate 30 and sandwiches the subject H between the subject platform 4 and the pressure plate 30 with a predetermined pressure, for example, 100 [N] when the subject H is placed on the subject platform 4. Here, at this time, the control unit 60 controls the pressure plate 30 in order not to have a pressure on the subject H more than a certain value.

Further, to the control device 60, input are the age of the examinee and the density and the thickness of the subject H among the above-mentioned control conditions from the input device 12 and the rail 31.

Further, the control device 60 increases the above-mentioned standard irradiation dose when the interval R1 between the radiation source 7 and the subject platform 4 is large for setting large irradiation dose of radiation to be irradiated. Concretely, the control device 60 resets the above-mentioned standard irradiation dose as 50×(R1²/65²) [mAs], multiplies a value of the above-mentioned standard irradiation dose by a value corresponding to the input control condition among the values in the first to third data tables and sets a result of the calculation as irradiation dose. Here, at this time, the control device 60 may use focus diameter information stored in the memory 47 in advance, radiation intensity information from the radiation image detectors 23, 26 and 29, radiography mode information or the like for the above-mentioned calculation.

Further, the control device 60 controls irradiation through a radiation source controller 55 based on the set irradiation dose. At this time, the control device 60 controls the radiation source 7 so as to make exposure dose of the subject H not more than 3 [mGy]. Here, since it is preferable that upper limit of the above-mentioned exposure dose be small, the upper limit is not limited to 3 [mGy]. For example, the upper limit may be set as 2 [mGy], or even smaller a value. As a method for changing exposure dose of the subject H, a method for changing an irradiation period with irradiation dose per unit of time fixed, and a method for changing irradiation dose per unit of time can be cited.

Further, the control device 60 outputs the above-mentioned control conditions to the monitor 13, the image output device 54 or the like before an image is radiographed.

Further, if the same subject is to be radiographed, by use of the control device 60, it is possible to select whether to use the irradiation conditions which have been set to the subject in the past, or to reset the irradiation conditions newly. Concretely, if the control conditions have not been changed from the radiography in the past, the control device 60 uses the past irradiation conditions stored in the memory 47. If the control conditions have been changed, the control device 60 sets the irradiation conditions newly.

In the case of setting the irradiation conditions newly, when irradiation dose of radiation to be irradiated at this time is more than a predetermined ratio compared to that of radiation irradiated at the last time, 1.2 times more for example, the control device 60 notifies the operator accordingly via the monitor 13 or the like.

Further, in the case of using the past irradiation conditions, the control device 60 uses the newest ones, that is, the last ones among the past irradiation conditions.

Next, operation of the mammography apparatus 1 of the present invention at the time of performing radiography will be explained.

First, when the subject H is placed on the subject platform 4, the pressure plate 30 is lowered so as to compress the subject H. Thereafter, the controller 11 determines a focus size of the radiation source 7 according to the radiography mode set by the switcher 45. Concretely, a radiation tube having the focus size of 100 [μm] is used in the phase contrast image radiography mode, and a radiation tube having the focus size of 300 [μm] is used in the normal radiography mode.

Then, the controller 11 recognizes each of the control conditions, resets the standard irradiation dose based on a value of the R1, sets the irradiation dose of the radiation source 7 with reference to the first data table, the second data table and the third data table, and then starts irradiating radiation. This gives, the radiation including subject information by transmitting through the subject H is irradiated to the radiation image detector.

According to the above-mentioned mammography apparatus 1, the controller 11 sets the irradiation conditions of radiation to be irradiated from the radiation source 7 based on the control conditions, in other words, the interval R1 between the radiation source 7 and the subject platform 4, the interval R3 between the under surface of the pressure plate 30 and the upper surface of the subject platform 4, thickness of the subject H or the like. Therefore, it is possible to estimate exposure dose of the subject H from the control conditions without using a phototimer and to set the irradiation conditions or radiation based on the estimation result. As mentioned above, since the irradiation conditions are set without using a phototimer unlike the conventional art, it is possible to surely prevent from irradiating radiation having so small irradiation dose that the radiation image detectors 23, 26 and 29 cannot detect the radiation, regardless of the position of the subject H on the subject platform 4. In other words, it is possible to irradiate radiation having enough irradiation dose to radiograph an image surely. Further, it is possible to prevent from irradiating excessive amount of radiation to the examinee.

Further, since the controller 11 sets the irradiation conditions of radiation to be irradiated from the radiation source 7 based on the interval R1 between the radiation source 7 and the subject platform 4, the thickness of the subject H and the age of the examinee, it is possible to estimate exposure dose to the subject H more accurately. Therefore, it is possible to irradiate radiation having enough irradiation dose to radiograph an image more surely.

Further, since the controller 11 sets the irradiation conditions of radiation to be irradiated from the radiation source 7 based on the interval R1 between the radiation source 7 and the subject platform 4, the thickness of the subject H and the density information of the subject H, it is possible to estimate exposure dose to the subject H more accurately. Therefore, it is possible to irradiate radiation having enough irradiation dose to radiograph an image more surely.

Further, since the rail 31 has a function as an ohmmeter, the thickness of the subject H can be measured as a distance from the under surface of the pressure plate 30 to the upper surface of the subject platform 4. Therefore, it is possible to prevent from the thickness of the subject H of the identical examinee changing depending on the operator, and thereby it is possible to measure the thickness of the subject H accurately. Therefore, it is possible to estimate exposure dose of the subject H more accurately and to irradiate radiation having enough irradiation dose to radiograph an image surely.

Further, since the controller 11 uses the measurement result by the measuring device when the subject H is sandwiched with a predetermined pressure as the thickness of the subject H, it is possible to prevent from the thickness of the subject H of the identical examinee changing depending on the operator surely, and thereby it is possible to measure the thickness of the subject H more accurately. Therefore, it is possible to estimate exposure dose of the subject H more accurately, and to irradiate radiation having enough irradiation dose to radiograph an image surely.

Further, since the controller 11 comprises the first data table, the second data table and the third data table storing each of the irradiation conditions to be set and each of the control conditions correspondingly, and the irradiation conditions are set based on the control conditions with reference to these data tables, it is possible to estimate exposure dose of the subject H immediately, and to irradiate radiation having enough irradiation dose to radiograph an image.

Further, in the case of radiographing the identical subject H at the second time or more, when the irradiation conditions stored in the memory 47 in advance are used as the irradiation conditions of radiation to be irradiated for the radiography, radiation having the same irradiation dose is always irradiated to the identical subject H at any time of radiography. Therefore, since it is possible to stabilize irradiation dose to the identical subject H, it is possible to surely prevent from irradiating radiation having so small irradiation dose that the radiation image detector cannot detect the radiation. In other words, it is possible to irradiate radiation having enough irradiation dose to radiograph an image. Further, it is possible to prevent from irradiating excessive amount of radiation to the examinee.

Further, in the case of radiographing the identical subject H at the second time or more, when the irradiation conditions are to be set newly, if irradiation dose of radiation to be irradiated is more than a predetermined ratio compared to that of radiation irradiated at the past time, the operator is warned accordingly. Therefore, it is possible to prevent from irradiating excessive amount of radiation to the examinee.

Further, since the distance R1 from the radiation source 7 to the radiation image detector 26 is not less than 75 [cm] and the distance R2 from the subject platform 4 to the radiation image detector 26 is not less than 15 [cm], it is possible to radiograph a clear phase contrast image.

Further, since the distance R1 from the radiation source 7 to the radiation image detector 26 is not more than 200 [cm], it is possible to simplify the structure of the mammography apparatus.

Further, since the distance R2 from the subject platform 4 to the radiation image detector 26 is not more than 100 [cm] and therefore it is not too large compared to the distance R1 from the radiation source 7 to the subject platform 4, it is possible to radiograph a sharp image with little influence of unclearness.

Further, since a photostimulable phosphor plate is used as the radiation image detectors 23, 26 and 29, with the property of accumulative phosphor (photostimulable phosphor) emitting stimulated light according to energy of radiation irradiated to the subject H, it is possible to record radiation image information of the subject H and provide the image of the subject H.

Further, since a flat panel detector is used as the radiation image detectors 23, 26 and 29, it is possible to record radiation image information of the subject H by detecting intensity of radiation irradiated to the subject H, and provide the image of the subject H.

Further, since the focus size is not less than 50 [μm], it is possible to make amount of current at the radiation source 7 large. Therefore, since it is possible to obtain irradiation dose (=current amount×irradiation period) necessary to perform radiography despite irradiation for a short time period, it is possible to prevent from an image getting unclear due to the movement of organs in the examinee's body. In other words, it is possible to radiograph a sharp image with little unclearness.

Further, since the focus size is not more than 250 [μm], in the case of magnified radiography, unclearness of the half shadow hardly happens. Therefore, it is possible to radiograph a sharp image with little unclearness.

Incidentally, in the above-mentioned embodiment, what is explained is that the control device 60 controls the irradiation dose. However, the control device may control the tube voltage. Concretely, the control device 60 may control the tube voltage with reference to a fourth data table, a fifth data table, a sixth data table and a seventh data table as shown in Graphs 4 to 7 in the following. Here, the fourth to seventh data tables are to store relation between each of the control conditions and the tube voltage amount to be impressed to the radiation tube.

Graph 4:

| | distance | | |
|---|---|---|---|
| | 30 to 79 cm | 80 to 129 cm | 130 to 185 cm |
| tube voltage | 28 kVp | 30 kVp | 32 kVp |

Graph 5:

| | mamma thickness | | |
|---|---|---|---|
| | 3 cm and less | 3 to 5 cm | 5 cm and more |
| tube voltage | 26 kVp | 28 kVp | 30 kVp |

Graph 6:

| | density | | |
|---|---|---|---|
| | high | middle | low |
| tube voltage | 30 kVp | 28 kVp | 26 kVp |

Graph 7:

| | age | | | |
|---|---|---|---|---|
| | 30 and | | | 70 and |
| | lower | 30 s and 40 s | 50 s and 60 s | higher |
| tube voltage | 30 kVp | 29 kVp | 28 kVp | 26 kVp |

Further, the control device 60 may control the irradiation dose when the interval R1 between the radiation source 7 and the subject platform 4 is changed from 65 [cm], or the control device 60 may change the tube voltage with reference to the above-mentioned fourth data table, fifth data table, sixth data table and seventh data table when the interval R3 between the pressure plate 30 and the subject platform 4, the age of the examinee and the density of the subject H are respectively changed from the standard conditions.

Further, what is explained is that the subject H is sandwiched by moving the pressure plate 30. However, the subject H may be sandwiched by moving the subject platform 4.

Further, what is explained is that the interval R3 between the pressure plate 30 and the subject platform 4 is measured by use of the function of the rail 31 as an ohmmeter. However, the interval R3 between the pressure plate 30 and the subject platform 4 may be measured by detecting a groove or a projection which are placed on the rail 31. Further, what is explained is that the interval R3 between the pressure plate 30 and the subject platform 4 is measured with the rail 31. However, it may be measured with photometry by use of infrared rays.

Further, what is explained is that the input device 12 is placed separately from the controller 11. However, the input device 12 is not limited to the explanation above, and the input device 12 may be integrated with the controller 11. Further, what is explained is that the radiation source controller 55 is placed separately from the controller 11. However, the radiation source controller 55 is not limited to the explanation above, and the radiation source controller 55 may be integrated with the controller 11. Further, what is explained is that the input device 12 and the radiation source controller 55 are connected to the controller 11 as separate entities to each other. However, they may be integrated with each other to be connected to the controller 11.

The entire disclosure of Japanese Patent Application No. Tokugan 2002-378937 filed on Dec. 27, 2002 including specification, claims, drawings and summary are incorporated herein by reference in its entirety.

What is claimed is:

1. A mammography apparatus comprising:
a radiation source;
a subject platform for supporting a subject so as to face the subject to the radiation source;
a radiation image detector placed so as to be faced to the radiation source with respect to the subject platform for detecting radiation transmitted through the subject;
a controller for controlling the radiation source;
wherein the controller sets an irradiation condition of the radiation to be irradiated from the radiation source based on control conditions including at least thickness of the subject and a distance from the radiation source to the subject platform.

2. The apparatus of claim 1, wherein the control conditions include an age of an examinee.

3. The apparatus of claim 2, wherein
an age input device for inputting the age of the examinee is connected to the controller, and
the age input device outputs the input age to the controller.

4. The apparatus of claim 1, wherein the control conditions include density information regarding density of the subject.

5. The apparatus of claim 4, wherein
a density information input device for inputting the density information is connected to the controller, and
the density information input device outputs the input density information to the controller.

6. The apparatus of claim 1 further comprising:
a pressure plate for compressing the subject supported by the subject platform in conjunction with the subject platform,
a measuring device for measuring a distance from an under surface of the pressure plate to an upper surface of the subject platform,
wherein the controller uses a result of the measurement by the measuring device as the thickness of the subject.

7. The apparatus of claim 6, wherein the thickness of the subject is the result of the measurement by the measuring device when the pressure plate compresses the subject in conjunction with the subject platform with a predetermined pressure.

8. The apparatus of claim 1, wherein the controller comprises a first storage section for correspondingly storing the irradiation condition to be set and each of the control conditions as a data table in advance, and sets the irradiation condition by referring to the data table based on the control conditions.

9. The apparatus of claim 1, wherein the irradiation condition includes at least one of irradiation dose of the radiation and voltage impressed to the radiation source.

10. The apparatus of claim 1,
wherein the controller comprises a second storage section for correspondingly storing subject identification information for identifying the subject and the set irradiation condition, and
the controller uses the irradiation condition stored in the second storage section in advance as the irradiation condition of the radiation to be irradiated when the identical subject is to be radiographed at the second time or more.

11. The apparatus of claim 1 further comprising a notifying section for notifying an operator of abnormality of irradiation dose of the radiation to be irradiated,
wherein the controller comprises a second storage section for correspondingly storing subject identification information for identifying the subject and the set irradiation condition in advance, and is connected to the notifying section, and
when the identical subject is to be radiographed at the second time or more and irradiation dose of the radiation to be irradiated based on the set irradiation condition is more than predetermined amount with respect to irradiation dose of the radiation based on the irradiation condition stored in the second storage section in advance, the controller has the notifying section notify the operator accordingly.

12. The apparatus of claim 1,
wherein a distance from the radiation source to the radiation image detector is settable from 75 cm to 200 cm, and
a distance from the subject platform to the radiation image detector is settable from 15 cm to 100 cm.

13. The apparatus of claim 12,
wherein the distance from the radiation source to the radiation image detector is settable from 100 cm to 160, and
the distance from the subject platform to the radiation image detector is settable from 25 cm to 80 cm.

14. The apparatus of claim 1, wherein the radiation image detector is a photostimulable phosphor plate or a flat panel detector.

15. The apparatus of claim 1, wherein a focus size of the radiation source is from 50 μm to 250 μm.
